(19) United States
(12) Patent Application Publication
(10) Pub. No.: US 2025/0170109 A1
(43) Pub. Date: May 29, 2025

(54) METHOD OF TREATING MALIGNANT TUMOR BY AZABICYCLIC COMPOUND

(71) Applicant: TAIHO PHARMACEUTICAL CO., LTD., Tokyo (JP)

(72) Inventor: Shuichi OHKUBO, Tsukuba-shi (JP)

(73) Assignee: TAIHO PHARMACEUTICAL CO., LTD., Tokyo (JP)

(21) Appl. No.: 19/031,773

(22) Filed: Jan. 18, 2025

Related U.S. Application Data
(63) Continuation of application No. 17/055,446, filed on Nov. 13, 2020.

(30) Foreign Application Priority Data
May 14, 2018 (WO) PCT/JP2018/018593

(51) Int. Cl. A61K 31/437; A61K 9/00; A61P 35/00

(52) U.S. Cl. CPC A61K 31/437; A61K 9/0053; A61P 35/00

(57) ABSTRACT

Provided is a method of treating malignant tumor by an azabicyclic compound, particularly, with eye disorder reduced. The present invention provides a method for treating malignant tumor, comprising administering an effective amount of 3-ethyl-4-[3-(1-methylethyl)-4-[4-(1-methyl-1H-pyrazol-4-yl)-1H-imidazol-1-yl]-1H-pyrazolo[3,4-b]pyridin-1-yl]benzamide (compound 1) or a salt thereof to a patient in need thereof according to a dosing regimen, wherein the dosing regimen comprises administering the compound 1 or the salt thereof at a dose from 40 mg/body/day to 240 mg/body/day in terms of the amount of the compound 1 for consecutive days, and then providing a withdrawal period of at least 2 days.

This application is a Continuation of U.S. application Ser. No. 17/055,446, filed Nov. 13, 2020, pending, which is a 371 of of PCT/JP2019/019010, filed May 14, 2019, and claims benefit of Japanese Application No. PCT/JP2018/018593, filed May 15, 2018. The contents of each of these applications are incorporated herein by reference in their entirety.

### TECHNICAL FIELD

The present invention relates to a method of treating malignant tumor by an azabicyclic compound which has an HSP90 inhibitory effect and exhibits antitumor activity.

### BACKGROUND ART

A group of proteins called molecular chaperones have multiple functions in regulating other proteins such as promoting and maintaining the functional structure, promoting correct assembly, inhibiting unnecessary aggregation, protecting against degradation, and promoting secretion (Non-Patent Literature 1). HSP90 is a molecular chaperone present as abundantly as from approximately 1 to 2% of all intracellular soluble proteins, and unlike the other molecular chaperones, is not required for the biosynthesis of a majority of polypeptides (Non-Patent Literature 1). Signal transduction-related factors (e.g., ERBB1/EGFR, ERBB2/HER2, MET, IGF1R, KDR/VEGFR, FLT3, ZAP70, KIT, CHUK/IKK, BRAF, RAF1, SRC, and AKT), cell-cycle regulators (e.g., CDK4, CDK6, Cyclin D, PLK1, and BIRC5), and transcriptional factors (e.g., HIF-1α, p53, androgen receptor, estrogen receptor, and progesterone receptor) are known as major client proteins whose structural formation and stability are controlled through interaction with HSP90 (Non Patent Literatures 2 and 3). HSP90 is deeply involved in cell proliferation and survival by maintaining normal functions of these proteins. Furthermore, mutant or chimeric proteins which cause canceration and exacerbation of cancer (e.g., BCR-ABL and NPM-ALK) require HSP90 for their functions, indicating the importance of HSP90, particularly, for processes such as canceration and survival, proliferation, progression, and metastasis of cancer (Non-Patent Literature 2).

When functions of HSP90 as molecular chaperone are inhibited by its specific inhibitor such as geldanamycin, the inactivation, destabilization, and degradation of client proteins occur, resulting in the arrest of proliferation and apoptosis of cancer cells (Non-Patent Literature 4). From the viewpoint of physiological functions of HSP90, the HSP90 inhibitor is characterized by being able to inhibit multiple signaling pathways involved in the survival and proliferation of cancer at the same time. Therefore, the HSP90 inhibitor is capable of serving as a drug having a wide range of effective anticancer effects. HSP90 derived from cancer cells has higher activity and has higher affinity for ATP or its inhibitor than those of HSP90 derived from normal cells. From this finding, the HSP90 inhibitor is expected as a drug having high cancer selectivity (Non-Patent Literature 5).

The present applicant has reported in Patent Literature 1 that an azabicyclic compound mentioned below has high HSP90 inhibitory activity and antiproliferative effect on cancer cell lines, and is useful as an HSP90 inhibitor.

However, in previous clinical trials of HSP90 inhibitors, the occurrence of eye disorder such as night blindness, photopsia, or blurring of the eyes have often been observed as adverse events, though the degree differs among cases (Non-Patent Literature 7). The toxicity of HSP90 inhibitors to the eyes has also been observed in nonclinical models. Thus, it has been reported that the distribution of an HSP90 inhibitor in the retina correlates with ocular toxicity (Non-Patent Literature 6).

Thus, a method of treating malignant tumor using an HSP90 inhibitor which exhibits an excellent antitumor effect but exhibits no ocular toxicity is needed.

### CITATION LIST

### Patent Literature

Patent Literature 1: International Publication No. WO 2011/004610

### Non-Patent Literature

Non-Patent Literature 1: Nat. Rev. Cancer, 5:761-772 (2005)
Non-Patent Literature 2: TRENDS Mol. Med., 10:283-290 (2004)
Non-Patent Literature 3: Clin. Cancer Res., 15:9-14 (2009)
Non-Patent Literature 4: Curr. Opin. Pharmacol., 8:370-374 (2008)
Non-Patent Literature 5: Drug Resist. Updat., 12:17-27 (2009)
Non-Patent Literature 6: Mol. Cancer Ther., 14 (1), 14-22 (2015)
Non-Patent Literature 7: J. Clin. Oncol., 30 (26), 3277-3286 (2012)

### SUMMARY OF THE INVENTION

### Technical Problem

An object of the present invention is to provide a method of treating malignant tumor by an azabicyclic compound, particularly, with eye disorder reduced.

### Solution to Problem

The present inventor had conducted diligent studies to solve the problem and consequently revealed by studies using rats that an azabicyclic compound is eliminated within 24 hours from the rat retina, without exhibiting ocular toxicity, when administered for consecutive days (Non-Patent Literature 6). Therefore, the present inventor had planned to treat malignant tumor by administering the azabicyclic compound to humans for consecutive days. However, in actuality, the compound caused a side effect for vision impairment in the phase I clinical trial. Accordingly, as a result of conducting further studies, the present inventor has found that a specific method of administering an azabicyclic compound represented by the formula (I) described below or a salt thereof can treat malignant tumor with high effectiveness without causing severe eye disorder.

Specifically, the present invention provides the following inventions [1] to [39].

[1] A method for treating malignant tumor, comprising administering an effective amount of 3-ethyl-4-[3-(1-methylethyl)-4-[4-(1-methyl-1H-pyrazol-4-yl)-1H-imidazol-1-yl]-1H-pyrazolo[3,4-b]pyridin-1-yl]benzamide (compound 1) or a salt thereof to a patient in need thereof according to a dosing regimen, wherein the dosing regimen comprises administering the compound 1 or the salt thereof at a dose from 40 mg/body/day to 240 mg/body/day in terms of the amount of the compound 1 for consecutive days, and then providing a withdrawal period of at least 2 days.
[2] The method according to [1], wherein the withdrawal period is from 2 to 3 days.
[3] The method according to [1], wherein the withdrawal period is 2 days.
[4] The method according to any one of [1] to [3], wherein the compound 1 or the salt thereof is administered for 3 to 8 consecutive days.
[5] The method according to any one of [1] to [3], wherein the compound 1 or the salt thereof is administered for 4 to 7 consecutive days.
[6] The method according to any one of [1] to [3], wherein the compound 1 or the salt thereof is administered for 5 consecutive days.
[7] The method according to any one of [1] to [6], wherein the dose of the compound 1 or the salt thereof is from 80 mg/body/day to 200 mg/body/day.
[8] The method according to any one of [1] to [6], wherein the dose of the compound 1 or the salt thereof is from 120 mg/body/day to 160 mg/body/day.
[9] The method according to any one of [1] to [6], wherein the dose of the compound 1 or the salt thereof is 160 mg/body/day.
[10] The method according to any one of [1] to [9], wherein the malignant tumor is gastrointestinal stromal tumor, leukemia, breast cancer, lung cancer, stomach cancer, prostate cancer, ovary cancer and/or large intestinal cancer.
[11] The method according to any one of [1] to [9], wherein the malignant tumor is gastrointestinal stromal tumor, breast cancer and/or lung cancer.
[12] The method according to any one of [1] to [9], wherein the malignant tumor is gastrointestinal stromal tumor.
[13] The method according to any one of [1] to [11], wherein eye disorder is reduced.
[14] A therapeutic agent for malignant tumor, comprising 3-ethyl-4-[3-(1-methylethyl)-4-[4-(1-methyl-1H-pyrazol-4-yl)-1H-imidazol-1-yl]-1H-pyrazolo[3,4-b]pyridin-1-yl]benzamide (compound 1) or a salt thereof, wherein
the compound 1 or the salt thereof is administered according to a dosing regimen comprising administering the compound 1 or the salt thereof at a dose from 40 mg/body/day to 240 mg/body/day in terms of the amount of the compound 1 for consecutive days, and then providing a withdrawal period of at least 2 days.
[15] The therapeutic agent according to [14], wherein the withdrawal period is from 2 to 3 days.
[16] The therapeutic agent according to [14], wherein the withdrawal period is 2 days.
[17] The therapeutic agent according to any one of [14] to [16], wherein the compound 1 or the salt thereof is administered for 3 to 8 consecutive days.
[18] The therapeutic agent according to any one of [14] to [16], wherein the compound 1 or the salt thereof is administered for 4 to 7 consecutive days.
[19] The therapeutic agent according to any one of [14] to [16], wherein the compound 1 or the salt thereof is administered for 5 consecutive days.
[20] The therapeutic agent according to any one of [14] to [19], wherein the dose of the compound 1 or the salt thereof is from 80 mg/body/day to 200 mg/body/day.
[21] The therapeutic agent according to any one of [14] to [19], wherein the dose of the compound 1 or the salt thereof is from 120 mg/body/day to 160 mg/body/day.
[22] The therapeutic agent according to any one of [14] to [19], wherein the dose of the compound 1 or the salt thereof is 160 mg/body/day.
[23] The therapeutic agent according to any one of [14] to [22], wherein the malignant tumor is gastrointestinal stromal tumor, leukemia, breast cancer, lung cancer, stomach cancer, prostate cancer, ovary cancer and/or large intestinal cancer.
[24] The therapeutic agent according to any one of [14] to [22], wherein the malignant tumor is gastrointestinal stromal tumor, breast cancer and/or lung cancer.
[25] The therapeutic agent according to any one of [14] to [22], wherein the malignant tumor is gastrointestinal stromal tumor.
[26] The therapeutic agent according to any one of [14] to [25], wherein eye disorder is reduced.
[27] Use of 3-ethyl-4-[3-(1-methylethyl)-4-[4-(1-methyl-1H-pyrazol-4-yl)-1H-imidazol-1-yl]-1H-pyrazolo[3,4-b]pyridin-1-yl]benzamide (compound 1) or a salt thereof for producing a therapeutic agent for malignant tumor, wherein
the compound 1 or the salt thereof is administered according to a dosing regimen comprising administering the compound 1 or the salt thereof at a dose from 40 mg/body/day to 240 mg/body/day in terms of the amount of the compound 1 for consecutive days, and then providing a withdrawal period of at least 2 days.
[28] The use according to [27], wherein the withdrawal period is from 2 to 3 days.
[29] The use according to [27], wherein the withdrawal period is 2 days.
[30] The use according to any one of [27] to [29], wherein the compound 1 or the salt thereof is administered for 3 to 8 consecutive days.
[31] The use according to any one of [27] to [29], wherein the compound 1 or the salt thereof is administered for 4 to 7 consecutive days.
[32] The use according to any one of [27] to [29], wherein the compound 1 or the salt thereof is administered for 5 consecutive days.
[33] The use according to any one of [27] to [32], wherein the dose of the compound 1 or the salt thereof is from 80 mg/body/day to 200 mg/body/day.
[34] The use according to any one of [27] to [32], wherein the dose of the compound 1 or the salt thereof is from 120 mg/body/day to 160 mg/body/day.
[35] The use according to any one of [27] to [32], wherein the dose of the compound 1 or the salt thereof is 160 mg/body/day.
[36] The use according to any one of [27] to [35], wherein the malignant tumor is gastrointestinal stromal tumor, leukemia, breast cancer, lung cancer, stomach cancer, prostate cancer, ovary cancer and/or large intestinal cancer.
[37] The use according to any one of [27] to [35], wherein the malignant tumor is gastrointestinal stromal tumor, breast cancer and/or lung cancer.
[38] The use according to any one of [27] to [35], wherein the malignant tumor is gastrointestinal stromal tumor.
[39] The use according to any one of [27] to [38], wherein eye disorder is reduced.

### Effects of the Invention

The method of the present invention is capable of treating malignant tumor by exerting an excellent antitumor effect while reducing eye disorder.

### BRIEF DESCRIPTION OF DRAWINGS

FIG. 1 is a diagram showing results of a histopathological test on compound 1 and NVP-AUY992 in rat eye tissues.

FIG. 2 is a diagram showing quantitative results of plasma, tumor and retinal tissues exposed to compound 1 by oral administration and NVP-AUY992 by intravenous administration.

FIG. 3 is a diagram showing quantitative results of plasma and retinal tissues exposed to compound 1 by intravenous administration.

### DETAILED DESCRIPTION OF THE INVENTION

3-Ethyl-4-[3-(1-methylethyl)-4-[4-(1-methyl-1H-pyrazol-4-yl)-1H-imidazol-1-yl]-1H-pyrazolo[3,4-b]pyridin-1-yl]benzamide (hereinafter, also referred to as “compound 1”) of the present invention is represented by the following structural formula (I):

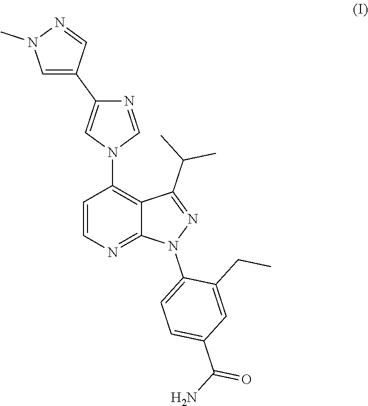 [SMILES: CCc1cc(C(N)=O)ccc1-n1nc(C(C)C)c2c(-n3cnc(-c4cnn(C)c4)c3)ccnc21]

The compound 1 or a salt thereof is a known compound and can be synthesized in accordance with a method described in, for example, Patent Literature 1 (International Publication No. WO 2011/004610).

The salt of the compound 1 is not particularly limited as long as the salt is pharmaceutically acceptable. Examples thereof include acid-addition salts with inorganic acids such as hydrochloric acid, hydrobromic acid, hydroiodic acid, sulfuric acid, nitric acid, and phosphoric acid, or organic acids such as formic acid, acetic acid, propionic acid, oxalic acid, malonic acid, succinic acid, fumaric acid, maleic acid, lactic acid, malic acid, citric acid, tartaric acid, carbonic acid, picric acid, methanesulfonic acid, p-toluenesulfonic acid, and glutamic acid, salts with inorganic bases such as sodium, potassium, magnesium, calcium, and aluminum, organic bases such as methylamine, ethylamine, meglumine, and ethanolamine, or basic amino acids such as lysine, arginine, and ornithine, and ammonium salts.

The compound 1 also encompasses hydrates, various solvates and crystal polymorphs.

In the present invention, the compound 1 or the salt thereof is administered in its effective amount to a patient in need of treatment of malignant tumor according to a dosing regimen.

In the present invention, the “effective amount” refers to a dose at which the compound 1 or the salt thereof to be administered exhibits an efficacy.

In the present invention, the dose of the compound 1 or the salt thereof per human per day on an administration day is from 40 mg/body/day to 240 mg/body/day in terms of the amount of the compound 1, or this dose is more preferably from 80 mg/body/day to 200 mg/body/day, further preferably from 120 mg/body/day to 160 mg/body/day, most preferably 160 mg/body/day. This dose may be administered in divided portions in one day.

In the present invention, the compound 1 or the salt thereof is administered according to a dosing regimen comprising administering the compound 1 or the salt thereof at the dose for consecutive days, and then providing a withdrawal period for which the compound 1 or the salt thereof is not administered to the patient for at least 2 days. In rats, the plasma concentration and retinal concentration of the compound 1 make a parallel transition. Therefore, as shown in Examples mentioned below, a withdrawal period (plasma concentration decreases to 5% or less of the highest plasma concentration in the duration of administration) of a period for which the plasma concentration of the compound 1 decreases drastically, i.e., at least 2 days, is provided during each administration for consecutive days. This withdrawal period is expected to remove the compound 1 from the retina. Therefore, the dosing regimen is expected to reduce eye disorder caused by the compound 1 or the salt thereof.

The withdrawal period is preferably from 2 to 3 days, particularly preferably 2 days.

The compound 1 or the salt thereof is preferably administered for 3 to 8 consecutive days, more preferably for 4 to 7 consecutive days, particularly preferably for 5 consecutive days.

In the present invention, the dosing regimen preferably comprises administering the compound 1 or the salt thereof for 3 to 8 consecutive days, and then providing a withdrawal period from 2 to 3 days, more preferably comprises administering the compound 1 or the salt thereof for 4 to 7 consecutive days, and then providing a withdrawal period from 2 to 3 days, further preferably comprises administering the compound 1 or the salt thereof for 4 consecutive days, and then providing a withdrawal period of 3 days, or administering the compound 1 or the salt thereof for 5 consecutive days, and then providing a withdrawal period of 2 days, and even more preferably comprises administering the compound 1 or the salt thereof for 5 consecutive days, and then providing a withdrawal period of 2 days. The cycle of this dosing regimen is preferably repeated as many times as possible.

In the present invention, the compound 1 or the salt thereof is most preferably administered according to a dosing regimen comprising administering the compound 1 or the salt thereof at 160 mg/body/day as a dose per human per day on an administration day for 5 consecutive days, and then providing a withdrawal period of 2 days.

In the present invention, specific examples of the malignant tumor to be treated include epithelial cancer (respiratory cancer, digestive system cancer, genitourinary cancer, secretory system cancer, skin cancer, etc.), mesothelioma, breast cancer, sarcoma, hematopoietic organ tumor, central nervous system tumor, and peripheral nervous system tumor. Among them, sarcoma is preferred.

Examples of the respiratory cancer include lung cancer (non-small cell lung cancer, small-cell lung cancer, etc.). Examples of the digestive system cancer include esophageal cancer, stomach cancer, duodenal cancer, liver cancer, biliary tract cancer (gallbladder cancer and bile duct cancer, etc.), pancreatic cancer, large intestinal cancer, and colorectal cancer (colon cancer, rectum cancer, etc.). Examples of the genitourinary cancer include ovary cancer, uterine cancer (uterine cervical cancer, uterine body cancer, etc.), kidney cancer, bladder cancer, prostate cancer, and testicular tumor. Examples of the secretory system cancer include neuroendocrine tumor. Examples of the mesothelioma include pleural mesothelioma, peritoneal mesothelioma, pericardial mesothelioma, and testicular mesothelioma. Examples of the sarcoma include gastrointestinal stromal tumor, bone tumor, and soft tissue tumor. Examples of the hematopoietic organ tumor include leukemia, malignant lymphoma, and multiple myeloma. Examples of the central nervous system tumor include brain tumor. Examples of the peripheral nervous system tumor include malignant neurilemmoma. Among them, gastrointestinal stromal tumor, leukemia, breast cancer, lung cancer, stomach cancer, prostate cancer, ovary cancer and large intestinal cancer are preferred. The lung cancer is preferably non-small cell lung cancer. Among them, leukemia, gastrointestinal stromal tumor, breast cancer and lung cancer are particularly preferred, and gastrointestinal stromal tumor is most preferred.

The “treatment” of malignant tumor of the present invention encompasses postoperative adjuvant chemotherapy which is performed for the prevention of recurrence after surgical extirpation of tumor, and preoperative adjuvant chemotherapy which is performed beforehand for surgical extirpation of tumor.

For pharmaceutical use of the compound 1 or the salt thereof, various dosage forms may be adopted according to therapeutic purposes. Examples of the form can include oral agents (tablets, coated tablets, powders, granules, capsules, solutions, etc.), injections, suppositories, patches, and ointments. An oral agent is preferred for the compound 1 or the salt thereof. These preparations can be formulated by common formation methods usually known in the art using a pharmaceutically acceptable carrier or the like.

Examples of the pharmaceutically acceptable carrier can include various additives generally used in usual drugs, for example, excipients, binders, disintegrants, lubricants, diluents, solubilizers, suspending agents, tonicity agents, pH adjusters, buffers, stabilizers, colorants, flavoring agents, and odor improving agents.

The preparation of the present invention comprising the compound 1 or the salt thereof may be a kit preparation comprising an instruction stating that the compound 1 or the salt thereof is administered according to a dosing regimen comprising administering the compound 1 or the salt thereof at a dose from 40 mg/body/day to 240 mg/body/day for consecutive days, and then providing a withdrawal period of at least 2 days. Specific examples of the “instruction” include package inserts and pamphlets. The kit preparation comprising the instruction may be a kit preparation comprising the instruction printed on or attached to a package, or may be a kit preparation comprising the instruction enclosed together with the compound 1 or the salt thereof in a package.

### EXAMPLES

Hereinafter, the present invention will be described in more detail with reference to Examples. However, the present invention is not limited by these Examples by any means. Various changes or modifications can be made by a person ordinarily skilled in the art without departing from the technical belief of the present invention.

### Example 1

Purpose: This study was conducted for the purpose of determining a maximum tolerated dose (MTD) and estimating a recommended dose (RD) and an administration method when compound 1 was administered with one cycle set to 21 days targeting solid cancer patients, and of evaluating safety and observing an antitumor effect when the compound 1 was administered with one cycle set to 21 days targeting the patients, for example.

Method: The compound 1 was repeatedly administered between meals once a day in Step 1, and orally administered between meals every other day in Step 2. In Step 1, the initial dose was set to 4.8 mg/m² (level 1), and the dose of the compound 1 at each level was determined depending on a body surface area. The initial dose of Step 2 (level 1 of Step 2) was set to MTD (mg/m²/day) of Step 1. The study was continued until the disease progressed or an intolerable adverse event occurred.

The dose (mg/body) to a study subject was calculated as described below.

Dose (mg/body)=Dose (mg/m²)×Body surface area (m²)

The body surface area (BSA) was calculated to the second decimal place by rounding a value off to two decimal places using the formula of DuBois given below. The dose (mg/body) was calculated to an integer by rounding a value off to the closest whole number, and less than 2 mg was cut off because the minimum tablet dose in this study was 2 mg.

BSA=([Body weight (kg)]0.425×[Body height (cm)]0.725)×0.007184

After determination of MTD, if the body surface area exhibited no relation to a pharmacokinetic parameter according to results of PK, the dose of an additional study after the determination of MTD was set to a fixed dose (mg/body) calculated with BSA as 1.62 m².

After determination of MTD for once-daily administration, administration was performed by once-daily administration, by alternate-day administration (repeated administration at cycles each involving one-day administration and one-day withdrawal period on the next day), or by a schedule involving administration for 5 consecutive days and 2-day withdrawal period, and evaluation was conducted.

An antitumor effect on tumor was determined according to RECIST Guideline Version 1.1 (European Journal of Cancer 45 (2009) 228-247), and tumor shrinkage was evaluated by comprehensive evaluation on target lesions (lesions equal to or larger than a measurable size depending on a slice width in a diagnosis method (MRI, CT, X ray, etc.) measured at the time of enrollment) and non-target lesions (all lesions except for the target lesions).

### Evaluation of Target Lesion

Target lesions were evaluated according to the following 5 levels.

### Complete Response (CR):

All non-lymph node target lesions disappeared, and the short axes of all lymph-node target lesions decreased to less than 10 mm (when lymph-node target lesions are selected before treatment, the target lesions may be evaluated as CR even if their sum axis is not 0 mm).

### Partial Response (PR):

The sum axis of target lesions decreased by 30% or more as compared with the sum before treatment.

### Stable Disease (SD):

Neither tumor shrinkage corresponding to CR or PR nor enlargement corresponding to PD was found.

### Progressive Disease (PD):

The sum axis of target lesions increased by 20% or more as compared with the previous smallest sum axis (including the sum before treatment), and the absolute value increased by 5 mm or more.

### Not Evaluable (NE):

Examination could not be performed for some reason, and all lesions or some lesions failed to be evaluated, or target lesions could not be evaluated as any of CR, PR, SD, and PD.

Even when the axis of a target lesion was determined to be “too small to measure” (e.g., 2 mm) or was less than 5 mm, an actual measurement value was recorded as much as possible. However, when a lesion was determined to no longer exist, the axis was recorded as 0 mm; and when a lesion was determined to remain, the axis was regarded as 5 mm.

### Evaluation of Non-Target Lesion

Non-target lesions were evaluated according to the following 4 levels.

### Complete Response (CR):

All non-lymph node non-target lesions disappeared, the short axes of all non-target lesions among lymph-node lesions decreased to less than 10 mm, and the levels of all tumor markers reached equal to or less than the institutional upper normal limits.

### Non-CR/Non-PD:

One or more non-lymph node non-target lesions did not disappear, or the short axes of one or more lymph-node non-target lesions reached 10 mm or more, or the level of any of tumor markers exceeded the institutional upper normal limits.

### Progressive Disease (PD):

### Evident Exacerbation of an Existing Non-Target Lesion

The “evident exacerbation” in the presence of a target lesion means that it is determined that treatment should be stopped due to increase in overall tumor mass even if an effect on the target lesion is SD or PR. Slight “exacerbation” based on a tumor mass does not correspond to the “evident exacerbation”. The evident exacerbation in the absence of a target lesion means, for example, increase in tumor mass corresponding to the enlargement of 73% of a tumor volume (which is equal to the enlargement of 20% of the axis of a measurable lesion).

### Not Evaluable (NE):

Examination could not be performed for some reason, and all lesions or some lesions failed to be evaluated, or non-target lesions could not be evaluated as any of CR, Non-CR/non-PD, and PD. However, even taken into account that the lesions which were not evaluated, if it was logically evident that any evaluated effect will not be changed, such a case was not counted as NE.

### Evaluation of Best Overall Response

The overall response of each case was evaluated according to the following criteria, and the best overall response was evaluated after confirmation of duration of the effect.

TABLE 1

| | | | |
|---|---|---|---|
| Overall response | Target lesion | Non-target lesion | New lesion |

Evaluation of overall response (in presence of target lesion)

| | | | |
|---|---|---|---|
| CR | CR | CR | Absent |
| PR | CR | Non-CR/non-PD | Absent |
| PR | CR | NE | Absent |
| PR | PR | Other than PD or NE | Absent |
| SD | SD | Other than PD or NE | Absent |
| NE | NE | Other than PD | Absent |
| PD | PD | Any level | Absent |
| | | | or Present |
| PD | Any level | PD | Absent |
| | | | or Present |
| PD | Any level | Any level | Present |

Evaluation of overall response (in absence of target lesion)

| | | |
|---|---|---|
| CR | CR | Absent |
| Non-CR/non-PD | Non-CR/non-PD | Absent |
| NE | NE | Absent |
| PD | PD | Absent |
| | | or Present |
| PD | Any level | Present |

The best overall response was evaluated according to the following 5 levels.
CR: After reaching CR first, the CR status was maintained in examination performed after a lapse of at least 4 weeks or longer.
PR: After reaching PR first, the PR status was maintained in examination performed after a lapse of at least 4 weeks or longer.
SD: Neither CR nor PR was attained and no PD was confirmed in examination performed after a lapse of at least 6 weeks or longer from the start of administration.
PD: PD of target lesions or non-target lesions was confirmed without satisfying the criterion of CR, PR and SD, or the appearance of a new lesion was confirmed.

If any of target lesions, non-target lesions, and the presence or absence of the appearance of new lesions were not evaluable, such a case was counted as NE.

### Response Rate (RR)

Proportion of cases with best overall response of CR and PR in an analyte population.

### Disease Control Rate (DCR)

Proportion of cases with best overall response of CR, PR, Non-CR/non-PD, or SD continued for 12 weeks or longer in an analyte population.

In this test, ophthalmologic examination was conducted in order to evaluate eye disorder.

The ophthalmologic examination was carried out before enrollment, in the duration of administration, and during follow-up. In the duration of administration, ophthalmologic interview, visual acuity examination (corrected), intraocular pressure examination, fundus examination, slit-lamp microscopy, optical coherence tomography (OCT), and color perception examination were carried out for 21 days±1 day from the start of administration. Observation and examination before enrollment were carried out as immediately before the enrollment as possible. In addition to the determined ophthalmologic examination described above, ophthalmologic examination was appropriately carried out when eye problems were found by regular interview by clinical investigators or by declaration from patients themselves.

The grading of the severity of adverse events which occurred in the eyes was evaluated according to the criteria of Common Terminology Criteria for Adverse Events (CTCAE Ver. 4.03) (Table 2).

TABLE 2

| CTCAE | | | | |
|---|---|---|---|---|
| v4.0 term | Grade 1 | Grade 2 | Grade 3 | Grade 4 |
| Eye | Asymptomatic or | Moderate; minimal, | Severe or medically | Sight-threatening |
| disorder | mild symptoms; | local or noninvasive | significant but not | consequences; |
| | clinical or diagnostic | intervention | immediately life- | urgent intervention |
| | observations only; | indicated; limiting | threatening; | indicated; best |
| | intervention not | age-appropriate | hospitalization or | corrected visual |
| | indicated | instrumental ADL | prolongation of | acuity or worse in |
| | | | hospitalization | the affected eye |
| | | | indicated; disabling; | (0.1 or less) |
| | | | limiting self care | |
| | | | ADL | |

Table 3 shows results of evaluating an antitumor effect on the tumors described above. Patients with non-small cell lung cancer, gastrointestinal stromal tumor, thymic cancer, biliary tract cancer, breast cancer, adenoid cystic cancer, pancreatic cancer, stomach cancer, ovary cancer, skin cancer, neuroblastoma, head and neck cancer, esophageal cancer, kidney cancer, colorectal cancer, sarcoma, mesothelioma, or the like entered the test.

TABLE 3
Results of tumor response in solid cancer based on
RECIST Guideline Version 1.1 for protocol set

| | QD | QD × 5 | QOD |
|---|---|---|---|
| | n = 16 (%) | n = 18 (%) | n = 26 (%) |
| PR | 2 (12.5) | 0 (0.0) | 1 (3.8) |
| SD | 7 (43.8) | 9 (50.0) | 9 (34.6) |
| PD | 5 (31.3) | 8 (44.4) | 15 (57.7) |
| NE | 2 (12.5) | 1 (5.6) | 1 (3.8) |
| RR | 2 (12.5) | 0 (0.0) | 1 (3.8) |
| DCR (≥12 weeks) | 5 (31.3) | 7 (38.9) | 4 (14.4) |

As a result of the study described above, MTD was 107.5 mg/m²/day for administration for consecutive days and 210 mg/m²/day for alternate-day administration.

The comparison of doses based on body surface areas with actual doses showed little influence on the correlation between the doses and the area under the plasma concentration-time curve (AUC) even if a method of calculating doses differed. Therefore, MTD was converted to the fixed dose (160 mg/body/day for administration for consecutive days and 340 mg/body/day for alternate-day administration), and an additional study was conducted.

Table 4 shows results of evaluating eye disorder in a consecutive-day administration group, an alternate-day administration group and a group given administration for 5 consecutive days and 2-day withdrawal period.

The degree of eye disorder at MTD in the group given administration for 5 consecutive days and 2-day withdrawal period and the alternate-day administration group was limited to grade 1, unlike the consecutive-day administration group.

This study was registered and conducted as NCT02965885 in ClinicalTrials.gov and JapicCTI-142444 in Japan Pharmaceutical Information Center.

### Example 2

Purpose: In order to evaluate the effectiveness and safety, etc. of compound 1, this study was conducted for the purpose of evaluating the effectiveness and safety, etc. of the compound 1, targeting patients with local progressive unresectable or metastatic GIST who received the administration of imatinib, sunitinib or regorafenib in the past.

From the results of the study of Example 1, this study was carried out according to a schedule involving administration for 5 consecutive days and 2-day withdrawal period. The daily dose of the compound 1 was 160 mg/body/day for oral administration. One cycle was set to 21 days. The administration of the compound 1 was continued until the end date of the clinical trial or any of predetermined criterion for discontinuation were applicable.

For the evaluation of an antitumor effect, CT scan was carried out before the start of administration, every 3 weeks for subsequent 6 weeks, every 6 weeks for subsequent 3 months, and then every 8 weeks until the end of the test. Progression-free survival (PFS) stipulated by RECIST Guideline Version 1.1 was evaluated.

Median PFS brought about by the compound 1 was 4.4 months. This result was favorable even when compared with the median PFS of 4.8 month in a group given the administration of regorafenib, a drug for the third-line treatment of progressive or unresectable GIST, or the median PFS of 0.9 months in a placebo administration group. The compound 1 also exhibited good tolerability. Although digestive tract disorder and elevated plasma creatinine levels were observed as related adverse events, all of these adverse events related to treatment were found to be reversible by the discontinuation of administration or decrease in dose and therefore considered to be controllable.

For the evaluation of eye disorder, interview, visual acuity examination (corrected), intraocular pressure measurement, fundus examination, slit-lamp microscopy, optical coherence tomography (OCT), and color perception examination were carried out at the time of a baseline within 14 days before enrollment and at the completion of administration of the study drug.

Interview, visual acuity examination (corrected), intraocular pressure measurement, fundus examination, optical coherence tomography (OCT), slit-lamp microscopy, and color perception examination were further carried out as much as possible every 12 weeks after the start of administration of the study drug and every 24 weeks on week 48 or later (within ±7 days).

In addition to the determined ophthalmologic examination described above, ophthalmologic examination was appropriately carried out when eye problems were found by regular interview by clinical investigators or by declaration from patients themselves.

The frequency of occurrence of eye disorder was 20%, and eye disorder in all the patients was at grade 1. This indicated the contribution of the dosing regimen of this test. These results suggested that the dosing regimen of administration for 5 consecutive days and 2-day withdrawal period is a suitable dosing regimen which exhibits an antitumor effect while reducing eye disorder.

This study was registered and conducted as JapicCTI-163182 in Japan Pharmaceutical Information Center.

### (Reference Example 1) Evaluation of Tissue Distribution Profile and Ocular Toxicity of Compound 1 in Rat—(1): Histopathological Study in Rat Eye Tissue

Method: Compound 1 was orally administered at 12 mg/kg/day to 6-week-old male Sprague-Dawley (SD) rats (Charles River Laboratories Japan, Inc., Japan) every day for 2 weeks. NVP-AUY922 (available from Funakoshi Co., Ltd., Japan) adjusted to 5% (v/v) dimethyl sulfoxide and 5% (v/v) Tween 20 in saline was intravenously administered at 10.0 mg/kg/day to SD rats three times a week for 2 weeks. The eyes were collected 24 hours after the final dose, fixed in a Davidson's solution over 48 hours, and preserved in a 10% (v/v) buffered formalin solution before predetermined paraffin embedding and slicing. The eye slices were stained with hematoxylin and eosin, followed by terminal deoxynucleotidyl transferase dUTP nick end labeling (TUNEL: ApopTagPlus Peroxidase In Situ Apoptosis Detection Kit, Merck-Millipore).

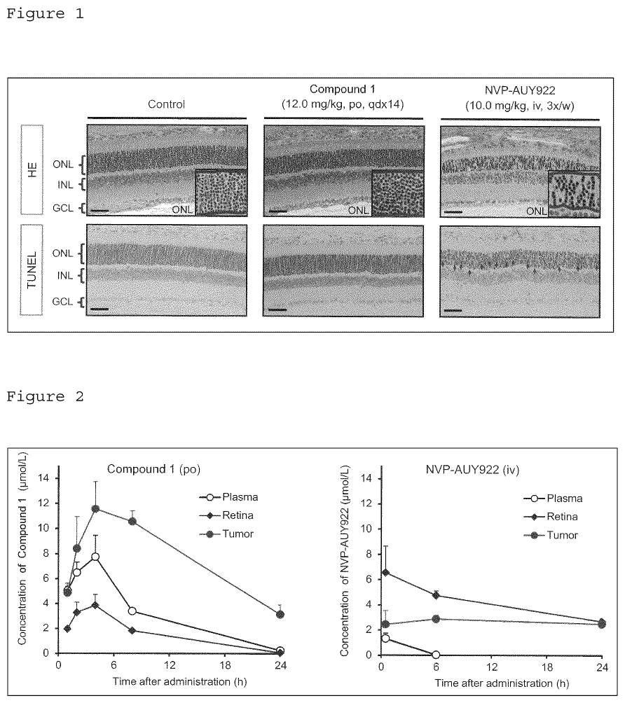

Results: The results are shown in FIG. 1. As is evident from these results, NVP-AUY922, another HSP90 inhibitor, was found to cause marked degeneration and disarrangement in the outer nuclear layer of the retina and to considerably increase the number of TUNEL-positive apoptotic cells ascribable to the retinal distribution of NVP-AUY922. On the other hand, the compound 1 did not cause any histopathological change in the outer nuclear layer of the retina, and such elevation of the number of apoptotic cells was not observed.

### (Reference Example 2) Evaluation of Tissue Distribution Profile and Ocular Toxicity of Compound 1 in Rat—(2): Quantification of Plasma, Tumor and Retinal Tissue Exposed to Compound 1 by Oral Administration

Method: Compound 1 and NVP-AUY922 were each administered to tumor-bearing F344 nude rats by oral or intravenous administration. After the administration, plasma, the retina, and tumor were collected according to a sampling schedule. The retina or tumor samples were homogenized in phosphate-buffered saline. The plasma and the homogenized samples were analyzed by liquid chromatography-tandem mass spectrometry or high-performance liquid chromatography. The pharmacokinetics of the compound 1 were calculated by use of the non-compartmental analysis method (Methods Mol Biol 2012; 929:377-89).

Results: The results are shown in FIG. 2. From these results, the compound 1 was found to distribute in a larger amount in tumor than in the retina and plasma in tumor-bearing rats. In the tumor-bearing rats, the compound 1 was confirmed to be removed from the retina within 24 hours. On the other hand, NVP-AUY922 distributed in a larger amount in the retina than in plasma and was detected from the retina even 24 hours after administration.

### (Reference Example 3) Evaluation of Tissue Distribution Profile and Ocular Toxicity of Compound 1 in Rat—(3): Quantification of Plasma and Retinal Tissue Exposed to Compound 1 by Intravenous Administration

Method: Compound 1 was administered to tumor-bearing F344 nude rats by intravenous administration. Plasma and the retina were collected 30 minutes and 6 hours after the administration. The retina samples were homogenized in phosphate-buffered saline. The plasma and the homogenized samples were analyzed by liquid chromatography-tandem mass spectrometry or high-performance liquid chromatography. The pharmacokinetics of the compound 1 were calculated by use of the non-compartmental analysis method (Methods Mol Biol 2012; 929:377-89).

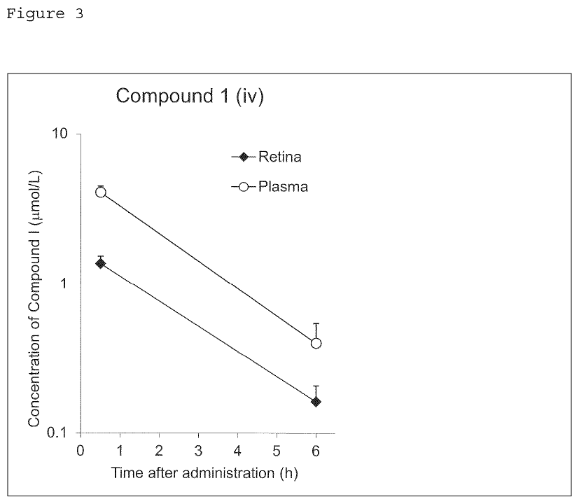

Results: The results are shown in FIG. 3. From these results, the distribution of the compound 1 was lower in the retina than in plasma, demonstrating that the distributions to both tissues show equilibrium transition respectively.

1. A method for treating malignant tumor, comprising administering an 3-ethyl-4-[3-(1-methylethyl)-4-[4-(1-methyl-1H-pyrazol-4-yl)-1H-imidazol-1-yl]-1H-pyrazolo[3,4-b]pyridin-1-yl]benzamide or a salt thereof (compound 1) to a patient in need thereof
at a dose of from 40 mg/body/day to 240 mg/body/day of compound 1 for at least one day, and then providing a withdrawal period of at least 2 days.

2. The method according to claim 1, wherein the withdrawal period is from 2 to 3 days.

3. The method according to claim 1, wherein the withdrawal period is 2 days.

4. The method of claim 1, wherein compound 1 or is administered for 3 to 8 consecutive days.

5. The method of claim 1, wherein compound 1 is administered for 4 to 7 consecutive days.

6. The method of claim 1, wherein the compound 1 is administered for 5 consecutive days.

7. The of claim 1, wherein the dose of the compound 1 is from 80 mg/body/day to 200 mg/body/day.

8. The method of claim 1, wherein the dose of the compound 1 is from 120 mg/body/day to 160 mg/body/day.

9. The method of claim 1, wherein the dose of the compound 1 is 160 mg/body/day.

10. The method of claim 1, wherein the malignant tumor is gastrointestinal stromal tumor, leukemia, breast cancer, lung cancer, stomach cancer, prostate cancer, ovary cancer and/or large intestinal cancer.

11. The method of claim 1, wherein the malignant tumor is gastrointestinal stromal tumor, breast cancer and/or lung cancer.

12. The method of claim 1, wherein the malignant tumor is gastrointestinal stromal tumor.

13. The method of claim 1, wherein the severity of eye disorder adverse events is reduced as compared to dosing regimens without at least one withdrawal period during the administration of compound 1.

14. A method of administering 3-ethyl-4-[3-(1-methylethyl)-4-[4-(1-methyl-1H-pyrazol-4-yl)-1H-imidazol-1-yl]-1H-pyrazolo[3,4-b]pyridin-1-yl]benzamide or a salt thereof (compound 1) which reduces adverse eye events when administering compound 1 to a subject with a malignant tumor, which comprises administering compound 1 at a dose from 40 mg/body/day to 240 mg/body/day of compound 1 for at least one day, followed a withdrawal period of at least 2 days to reduce adverse eye events.

15. The method of claim 14, wherein the withdrawal period is from 2 to 3 days.

16. The method of claim 14, wherein the withdrawal period is 2 days.

17. The method of claim 14, wherein the compound 1 is administered for 3 to 8 consecutive days.

18. The method of claim 14, wherein the compound 1 is administered for 4 to 7 consecutive days.

19. The method of claim 14, wherein the compound 1 is administered for 5 consecutive days.

20. The method of claim 14, wherein the dose of the compound 1 is from 80 mg/body/day to 200 mg/body/day.

21. The method of claim 14, wherein the dose of the compound 1 is from 120 mg/body/day to 160 mg/body/day.

22. The method of claim 14, wherein the dose of the compound 1 is 160 mg/body/day.

23. The method of claim 14, wherein the malignant tumor is gastrointestinal stromal tumor, leukemia, breast cancer, lung cancer, stomach cancer, prostate cancer, ovary cancer and/or large intestinal cancer.

24. The method of claim 14, wherein the malignant tumor is gastrointestinal stromal tumor, breast cancer and/or lung cancer.

25. The method of claim 14, wherein the malignant tumor is gastrointestinal stromal tumor.

26. The method of claim 14, wherein the severity of eye adverse events is reduced.
